(19) United States
(12) Patent Application Publication
(10) Pub. No.: US 2026/0294461 A1
(43) Pub. Date: Oct. 1, 2026

(54) SURGICAL BLADE ASSEMBLY

(71) Applicant: YorBo LLC, Utica, NY (US)

(72) Inventors: Jonathan D. Block, Whitesboro, NY (US); John A. Yorke, Utica, NY (US)

(21) Appl. No.: 19/576,685

(22) Filed: Mar. 24, 2026

Related U.S. Application Data
(60) Provisional application No. 63/777,672, filed on Mar. 25, 2025.

(51) Int. Cl. A61B 17/32; A61B 1/307; A61B 17/00

(52) U.S. Cl. CPC A61B 17/320016; A61B 1/307; A61B 2017/00367; A61B 2017/00743

(57) ABSTRACT

A surgical blade assembly for cutting a urethral stricture. The surgical blade assembly includes a handle defining a longitudinal axis extending centrally through the handle, a first recess extending along the axis, and a second recess extending from the first recess. A selector member is received in the handle and is movable between a locked state in which the selector member is received in the second recess and an unlocked state in which the selector member is received in the first recess. A sheath is coupled to the handle. A rod has a first end coupled to the selector member and a second end carrying a blade. The rod is at least partially surrounded by the sheath, which selectively covers the blade.

### CROSS-REFERENCE TO RELATED APPLICATIONS

The application claims priority to U.S. Provisional Patent Application No. 63/777,672, filed Mar. 25, 2025, the entire contents of which are incorporated by reference herein.

### FIELD

The present disclosure relates to the field of surgical care of urethral strictures, and tools and techniques for the effective treatment of such urological conditions.

### BACKGROUND

Urethral strictures and scars are a significant medical problem, causing urinary obstruction which can lead to decreased urinary flow, urinary retention, and a range of urinary symptoms. If left untreated, these conditions can result in poor bladder function or even renal failure. These strictures and scars, which can be caused by a variety of factors including trauma, infection, and surgical procedures, present a significant challenge in the field of urology.

### SUMMARY

In one embodiment, the present disclosure provides, in one aspect, a surgical blade assembly for cutting a urethral stricture, the surgical blade assembly comprising a handle defining a longitudinal axis that extends centrally through the handle. The handle includes a first recess that extends along the longitudinal axis and a second recess extending from the first recess. The surgical blade assembly includes a selector member received by the handle. The selector member is movable between a locked state in which the selector member is received in the second recess and an unlocked state in which the selector member is received in the first recess. The surgical blade assembly includes a sheath coupled to the handle. The surgical blade assembly includes a rod including a first end and a second end opposite from the first end. The first end is coupled to the selector member for movement therewith. The rod is at least partially surrounded by the sheath. The surgical blade assembly includes a blade disposed on the second end of the rod. The blade is selectively covered by the sheath.

In another embodiment, the present disclosure provides a method for cutting a urethral stricture, the method comprising inserting a sheath of a surgical blade assembly through an inlet port and into a working channel of a cystoscope, the surgical blade assembly including a handle coupled to the sheath and a blade disposed in the sheath and selectively covered by the sheath. The method includes inserting a sheath of the cystoscope into a urethra of a patient. The method includes identifying the stricture in the urethra through an ocular window of the cystoscope. The method includes deploying the blade from the sheath of the surgical blade assembly such that the blade engages the stricture.

In yet another embodiment, the present disclosure provides a surgical blade assembly configured to be partially received within a working channel of a cystoscope, the surgical blade assembly includes a handle defining a longitudinal axis that extends centrally through the handle. The surgical blade assembly includes a selector member received by the handle. The selector member is movable relative to the handle along the longitudinal axis. The surgical blade assembly includes a sheath fixedly coupled to the handle. The surgical blade assembly includes a rod at least partially surrounded by the sheath. The rod includes a first end and a second end opposite from the first end, the first end coupled to the selector member for movement therewith. The surgical blade assembly includes a blade formed on the second end of the rod. The blade is selectively covered by the sheath. The blade and the rod are formed from a single piece of material.

### BRIEF DESCRIPTION OF THE DRAWINGS

FIG. 1 is a side view of a known rigid cystoscope.

FIG. 2 is a schematic cross-sectional view of the rigid cystoscope of FIG. 1, taken along line 2-2 in FIG. 1.

FIG. 3 is a schematic cross-sectional view of the rigid cystoscope of FIG. 1, taken through a portion of a bridge and a sheath of the rigid cystoscope.

FIG. 4 is a schematic view of the rigid cystoscope of FIG. 1 inserted into a urethra of a patient.

FIG. 5 is a side view of a known urethrotome.

FIG. 6 is a side view of a known blade assembly for use with the urethrotome of FIG. 5.

FIG. 7 is a perspective view of a surgical blade assembly according to the present disclosure.

FIG. 8 is an exploded view of the surgical blade assembly of FIG. 7.

FIG. 9 is a perspective view illustrating a selector member of the surgical blade assembly of FIG. 7 in a locked state and an undeployed position.

FIG. 10 is a perspective view illustrating the selector member of the surgical blade assembly of FIG. 7 in an unlocked state and the undeployed position.

FIG. 11 is an enlarged perspective view of the surgical blade assembly of FIG. 7 taken along line 11-11 in the undeployed position.

FIG. 12 is a perspective view illustrating the selector member of the surgical blade assembly of FIG. 7 in the unlocked state and a deployed position.

FIG. 13 is a perspective view of the surgical blade assembly of FIG. 7 in the unlocked state and the deployed position.

FIG. 14 is an enlarged perspective view of the surgical blade assembly of FIG. 13 taken along line 14-14 in the deployed position.

FIG. 15A is a schematic end view of the surgical blade assembly of FIG. 7 in the unlocked state.

FIG. 15B is a schematic end view of the surgical blade assembly of FIG. 7 in the locked state.

FIG. 16 is a schematic view of the surgical blade assembly of FIG. 7 inserted into the rigid cystoscope of FIG. 1.

FIG. 17 is a schematic view of the surgical blade assembly of FIG. 7 in the locked state and the undeployed position with the rigid cystoscope of FIG. 1 inserted into the urethra of the patient.

FIG. 18 is a schematic view of the surgical blade assembly of FIG. 7 in the unlocked state and the undeployed position with the rigid cystoscope of FIG. 1 inserted into the urethra of the patient.

FIG. 19 is a schematic view of the surgical blade assembly of FIG. 7 in the unlocked state and the deployed position with the rigid cystoscope of FIG. 1 inserted into the urethra of the patient.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### DETAILED DESCRIPTION

The terms “substantially,” “essentially,” “approximately,” “about,” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “one of,” without a more limiting modifier such as “only one of,” and when applied herein to two or more subsequently defined options such as “one of A and B” should be construed to mean an existence of any one of the options in the list alone (e.g., A alone or B alone) or any combination of two or more of the options in the list (e.g., A and B together).

A device or structure that is “configured” in a certain way is configured in at least that way but may also be configured in ways that are not listed.

The terms “coupled,” “coupling,” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through intermediate elements or devices via an electrical element, electrical signal or a mechanical element depending on the particular context.

In the foregoing specification, specific embodiments are described. However, one of ordinary skill in the art appreciates that various modifications and changes may be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus that comprises, has, includes, contains the element. Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

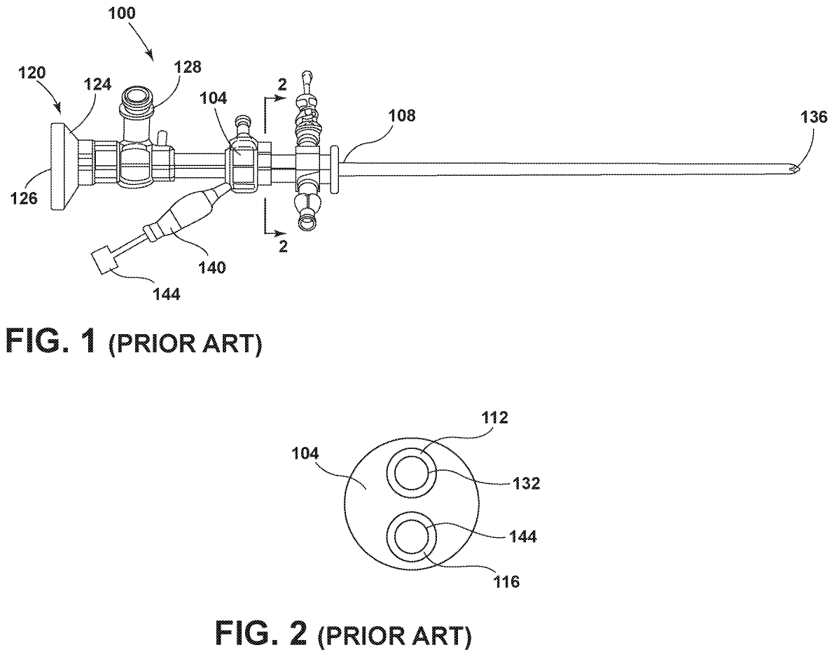

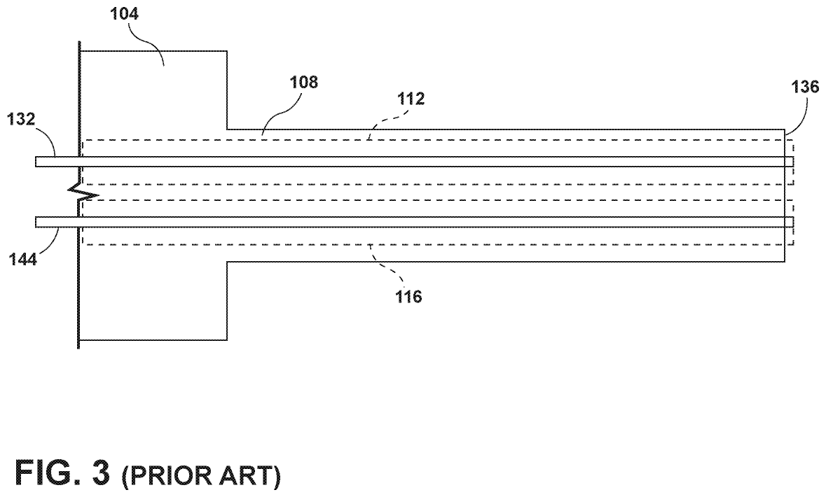

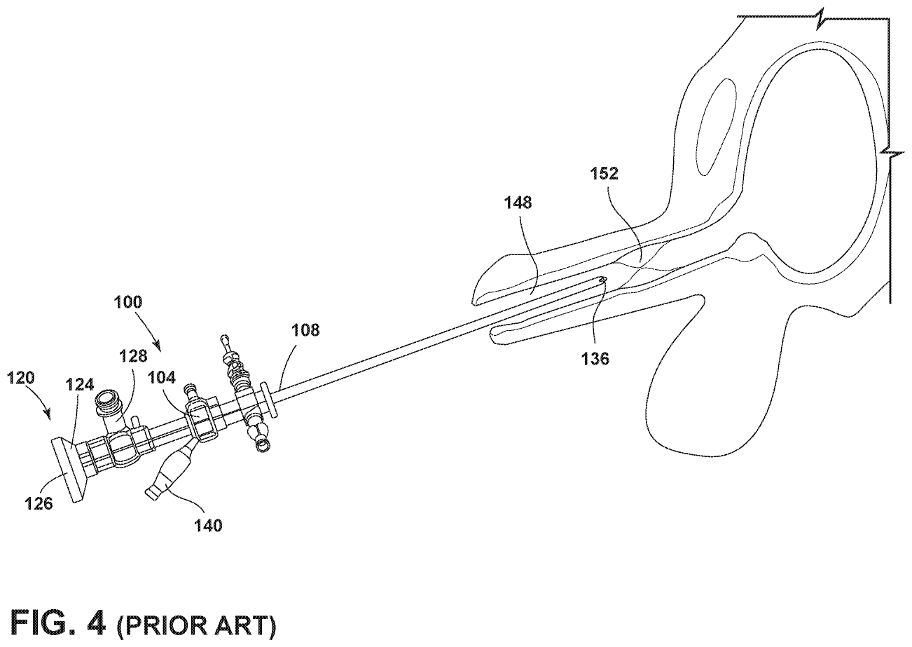

FIGS. 1-4 illustrate a known rigid cystoscope 100. The cystoscope 100 can be inserted into the urethra to directly view the urethra and bladder (see FIG. 4). The cystoscope 100 includes a bridge 104 and a sheath 108 extending from the bridge 104. The bridge 104 and the sheath 108 define a first channel 112 and a working channel 116 that is separate from the first channel 112. The cystoscope 100 includes an ocular assembly 120 that has an ocular funnel 124 that includes an ocular window 126 and a light pillar 128 that selectively receives a light source. The ocular assembly 120 includes an optical line 132 that relays an image from a beak 136 of the sheath 108. In the illustrated construction, the first channel 112 receives the optical line 132. The light pillar 128 provides light to the optical line 132 via the light source. Specifically, the light source provides light proximal to the beak 136. The cystoscope 100 includes an inlet port 140 of the working channel 116. In the illustrated construction, the inlet port 140 is offset by a non-zero angle relative to the portion of the working channel 116 in the sheath. In the illustrated construction, there is a single inlet port. In some constructions, there can be multiple inlet ports that are connected to the working channel 116. The working channel 116 selectively receives an accessory 144 through the inlet port 140 and the accessory 144 can be positioned at the beak 136. Examples of the accessory 144 include a stent placer or a stone basket. FIG. 3 illustrates that the optical line 132 and the accessory 144 extend through the bridge 104 and the sheath 108. Specifically, the optical line 132 and the accessory 144 are received at the beak 136. FIG. 4 illustrates the cystoscope 100 inserted into a urethra 148 of a patient without the accessory 144 in the working channel 116. In the position shown in FIG. 4, the beak 136 of the cystoscope 100 is positioned facing a urethral stricture 152 such that the healthcare provider can evaluate the stricture 152.

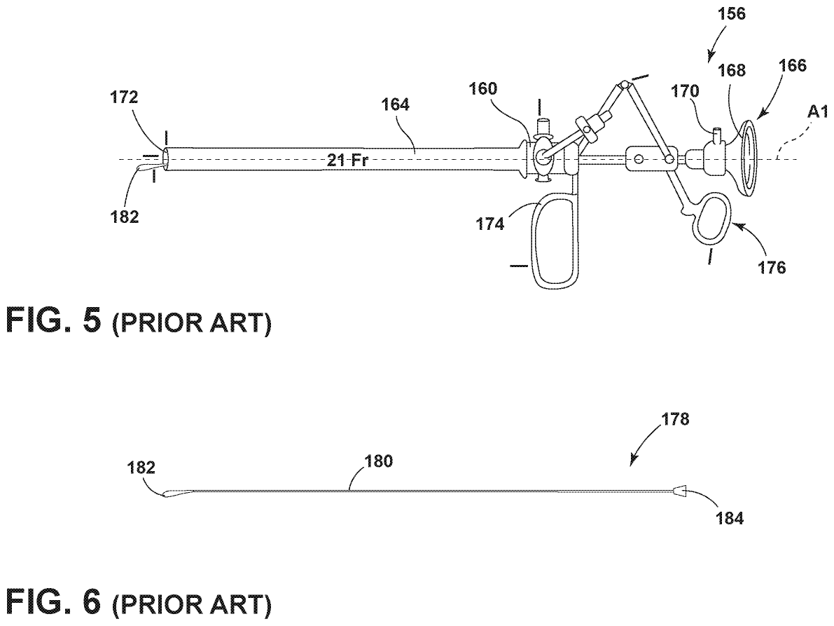

FIG. 5 illustrates a urethrotome 156 that is commonly used to treat the stricture 152. The urethrotome 156 includes a bridge 160 and a sheath 164 extending from the bridge 160. The urethrotome 156 includes an ocular assembly 166. Similar to the ocular assembly 120, the ocular assembly 166 includes an ocular funnel 168, a light pillar 170 that selectively receives a light source, and an optical line that is received at a beak 172 of the sheath 164. The urethrotome 156 includes a grip 174 that extends from the bridge 160 and is fixed thereto. The urethrotome 156 includes a working element 176 that is pivotably coupled to the bridge 160.

FIG. 6 illustrates a knife assembly 178 that is compatible with the urethrotome 156. The knife assembly 178 includes a rod or a wire 180, a blade 182 connected to one end of the wire 180, and a connecting end 184 disposed on the wire 180 at an opposite end from the blade 182. The connecting end 184 is coupled to the working element 176 such that the knife assembly 178 is movable within the sheath 164. The knife assembly 178 is a one-time-use device and is disposed of after each procedure. To couple the knife assembly 178 to the urethrotome 156, the connecting end 184 is inserted into the sheath 164 first and the connecting end 184 is coupled to the working element 176 through linkages. The knife assembly 178 is incapable of being used with the cystoscope 100. In some constructions, the rod or wire 180 is not flexible enough and cannot be passed through the inlet port 140 since the inlet port 140 is at an angle relative to the working channel 116. In some constructions, the blade 182 and/or the connecting end 184 are too large to be received by the inlet port 140 and/or the working channel 116. Also, the rigid cystoscope 100 does not include any actuators like the working element 176, so controlled and precise movement of the knife assembly 178 is difficult. Additionally, the knife assembly 178 does not include a dedicated sheath, and therefore the blade 182 would be susceptible to engaging the working channel 116 and degrading the edge of the blade 182. Accordingly, the urethrotome 156 was created to accommodate the knife assembly 178.

Referring back to FIG. 5, when inserting the urethrotome 156 into the urethra 148, the blade 182 is disposed within the sheath 164 such that the urethra 148 is not damaged. To engage the stricture 152, the working element 176 is moved in a direction parallel to a longitudinal axis A1, which extends longitudinally through the sheath 164, such that the blade 182 emerges from the sheath 164.

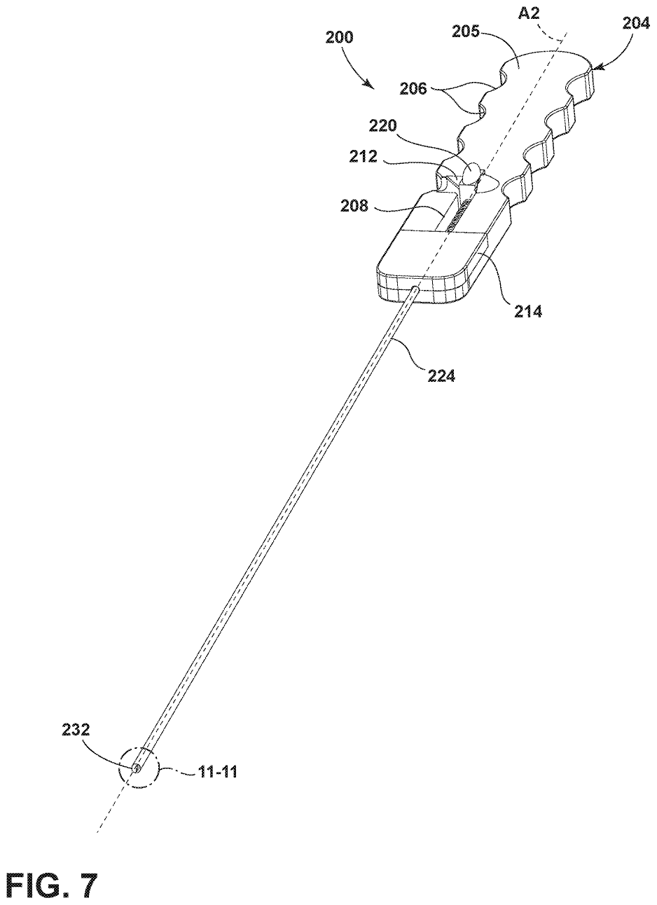

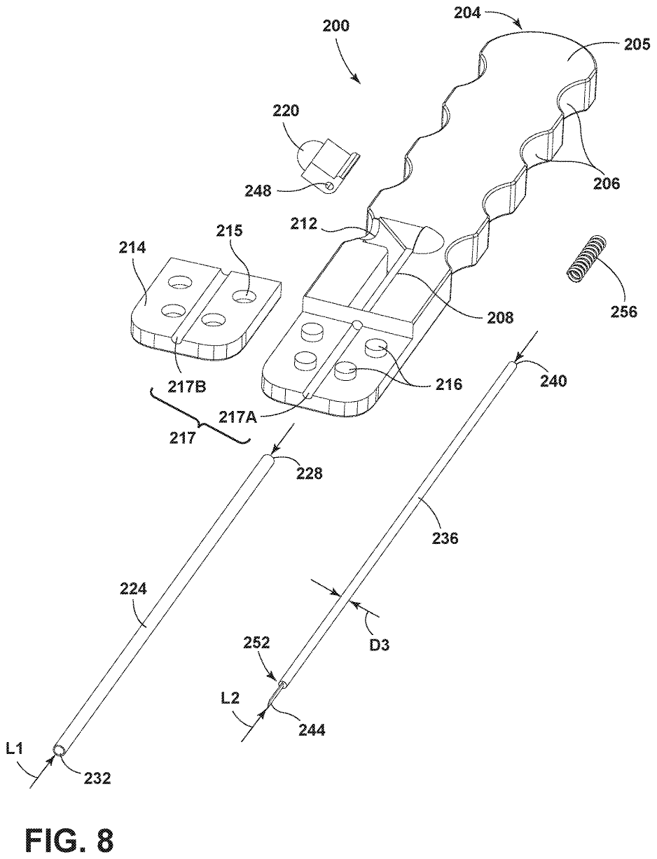

FIGS. 7 and 8 illustrate a surgical blade assembly 200, according to a construction of the present disclosure. The surgical blade assembly 200 is compatible with the cystoscope 100. The surgical blade assembly 200 includes a handle 204 that is intended to be received in a hand of a healthcare provider. The handle 204 includes a main body 205 having a plurality of grooves 206 to aid in gripping the handle 204. In some constructions, the handle 204 includes surface texturing to aid in gripping the handle 204. The handle 204 defines a longitudinal axis A2. In the illustrated construction, the longitudinal axis A2 extends centrally through the handle 204. The handle 204 includes a first recess 208 that extends along the longitudinal axis A2 and a second recess 212 extending from the first recess 208. The first recess 208 extends longer in a direction parallel to the longitudinal axis A2 than the second recess 212. The second recess 212 extends in a direction perpendicular to the longitudinal axis A2. In the illustrated construction, the first and the second recesses 208, 212 are defined in the main body 205. The handle 204 includes a removable portion 214 that is coupled to the main body 205. The removable portion 214 includes recesses 215 that are configured to receive protrusions 216 of the main body 205. The handle 204 defines a channel 217. In the illustrated construction, the channel 217 extends in a direction parallel to the longitudinal axis A2 and extends from an outside of the handle 204 to the first recess 208. In the illustrated construction, a first half 217A of the channel 217 is defined on the main body 205 and a second half 217B is defined on the removable portion 214. In the illustrated construction, the first half 217A and the second half 217B come together to form the channel 217.

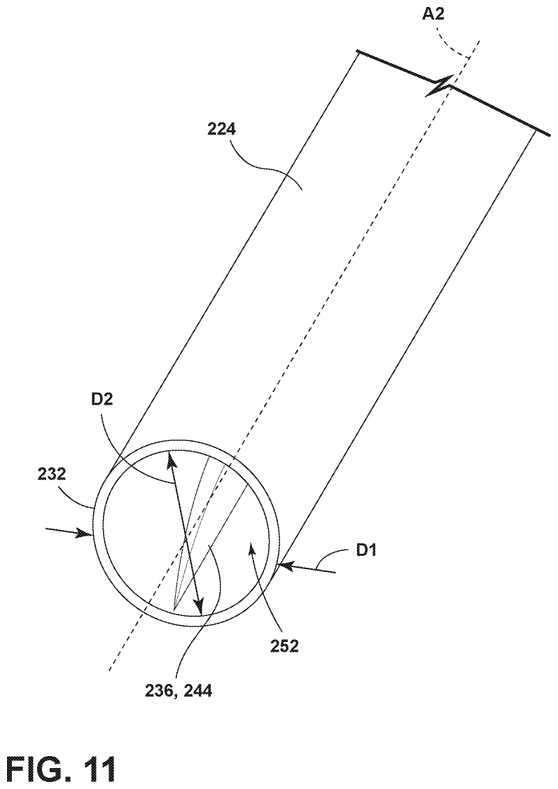

With continued reference to FIGS. 7 and 8, the surgical blade assembly 200 includes a selector member 220. In the illustrated construction, the selector member 220 is received by the handle 204. In the illustrated construction, the selector member 220 is selectively received either in the first recess 208 or in the second recess 212. The surgical blade assembly 200 includes a sheath 224 coupled to the handle 204. In the illustrated construction, the sheath 224 includes a first end 228 and a second end 232 opposite from the first end 228. The sheath 224 is a hollow tube that is received in the channel 217 such that the sheath 224 is fixed relative to the handle 204. In the illustrated construction, the first end 228 is received in the channel 217. In the illustrated construction, the sheath 224 is constructed from polyether block amide (e.g., Pebax). In the illustrated construction, the sheath 224 has a 72 D shore hardness. In the illustrated construction, the sheath 224 defines an outer diameter D1 of 2.18 millimeters (FIG. 11). In some constructions, the outer diameter D1 is between 1 millimeter and 4 millimeters. In the illustrated construction, the sheath 224 defines an inner diameter D2 of 1.93 millimeters (FIG. 11). In some constructions, the inner diameter D2 is between 0.75 millimeters and 3.75 millimeters. In the illustrated construction, the sheath 224 defines a length L1 measured from the first end 228 to the second end 232. In the illustrated construction, the length L1 is 406.4 millimeters. In some constructions, the length L1 is between 200 millimeters and 600 millimeters. An interior surface of the sheath 224 is smooth such that nothing is caught on an inside surface of the sheath 224.

With continued reference to FIGS. 7 and 8, the surgical blade assembly 200 includes a rod 236. In the illustrated construction, the outer surface of the rod 236 is cylindrical. In some constructions, the outer surface of the rod 236 defines a different shape (e.g., an octagon). The rod 236 includes a first end 240 and a second end 244 opposite from the first end 240. The rod 236 is at least partially received within the sheath 224. In other words, the rod 236 is at least partially surrounded by the sheath 224. The rod 236 is at least partially received by the handle 204. The rod 236 is coupled to the selector member 220 for movement therewith. In the illustrated construction, the rod 236 extends through the channel 217. In the illustrated construction, the first end 240 is coupled to the selector member 220. In the illustrated construction, the selector member 220 includes an aperture 248 that receives the first end 240. In the illustrated construction, the rod 236 is constructed from a nickel and titanium alloy (e.g., Nitinol). The rod 236 defines an outer diameter D3. In the illustrated construction, the diameter D3 is 1.6 millimeters. In some constructions, the diameter is between 0.6 millimeters and 3.5 millimeters. The rod 236 defines a length L2 measured between the first end 240 and the second end 244 in a direction parallel to the longitudinal axis. In the illustrated construction, the length L2 is 441.3 millimeters. In other constructions, the length is between 235 millimeters and 635 millimeters.

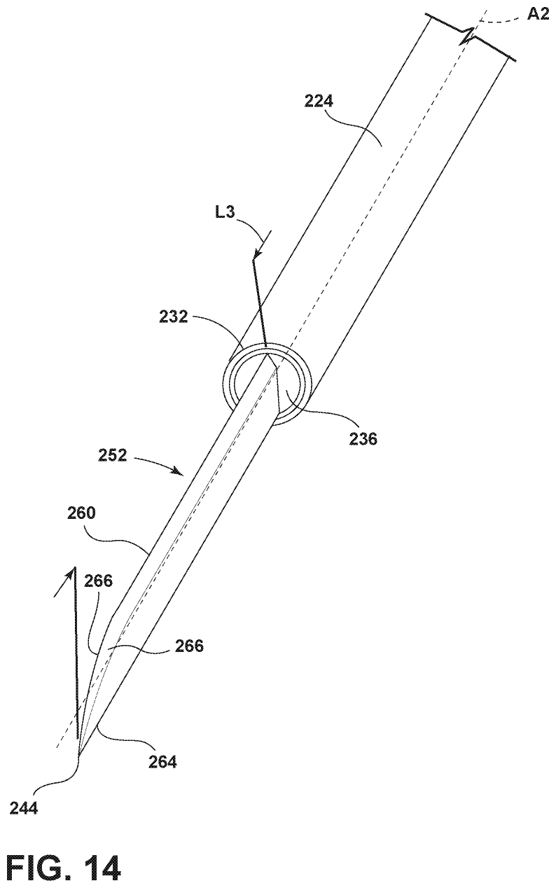

With continued reference to FIGS. 7 and 8, the surgical blade assembly 200 includes a blade 252. The blade 252 is disposed on the second end 244 of the rod 236. The blade 252 is selectively covered by the sheath 224. In the illustrated construction, the blade 252 and the rod 236 are formed from a single piece of material. That is, the blade 252 is formed on the rod 236 (e.g., monolithic construction). Additional details regarding the blade 252 will be described with respect to FIG. 14.

The surgical blade assembly 200 includes a spring 256. The spring 256 is received by the handle 204. In the illustrated construction, the spring 256 is disposed in the first recess 208 and surrounds a portion of the rod 236. The spring 256 is disposed between the main body 205 and the removable portion 214 and the selector member 220.

With reference to FIG. 7, when the surgical blade assembly 200 is assembled, the removable portion 214 is coupled to the main body 205 such that the channel 217 is defined therebetween. The sheath 224 is coupled to the handle 204 such that the sheath 224 does not move relative to the handle 204. In the illustrated construction, the sheath 224 is received between the main body 205 and the removable portion 214. The rod 236 is at least partially received in the sheath 224. In the illustrated construction, the first end 240 of the rod 236 is disposed nearer to the first end 228 of the sheath 224 than the second end 244. In the illustrated construction, the blade 252 is disposed nearer to the second end 232 of the sheath 224 than the first end 228. The first end 240 of the rod 236 is coupled with the selector member 220 for movement therewith, which is disposed in the first recess 208 or the second recess 212. The spring 256 is disposed around the rod 236 and between the selector member 220 and the handle 204.

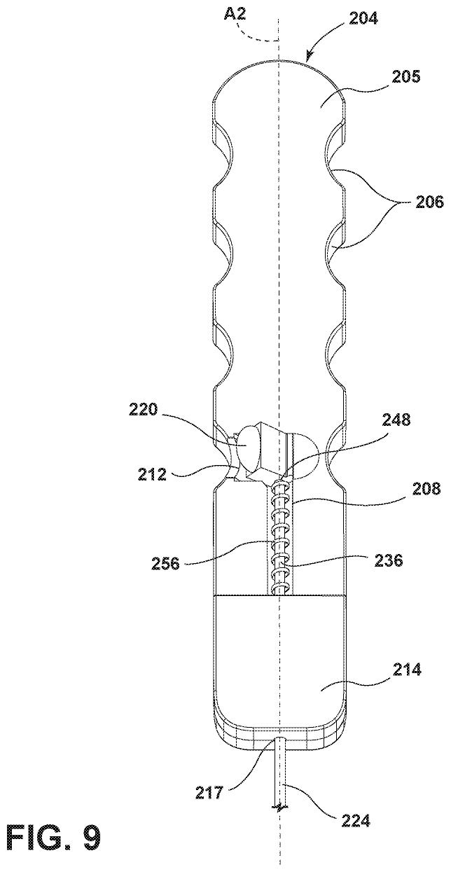

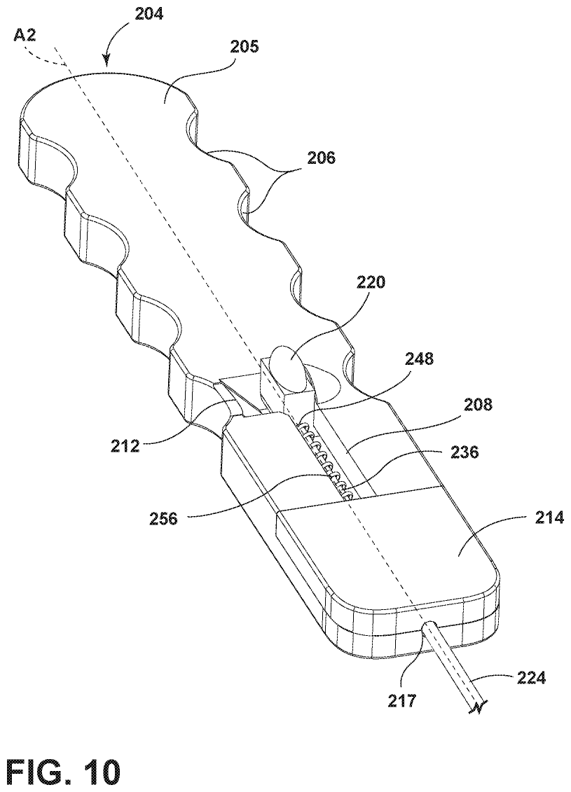

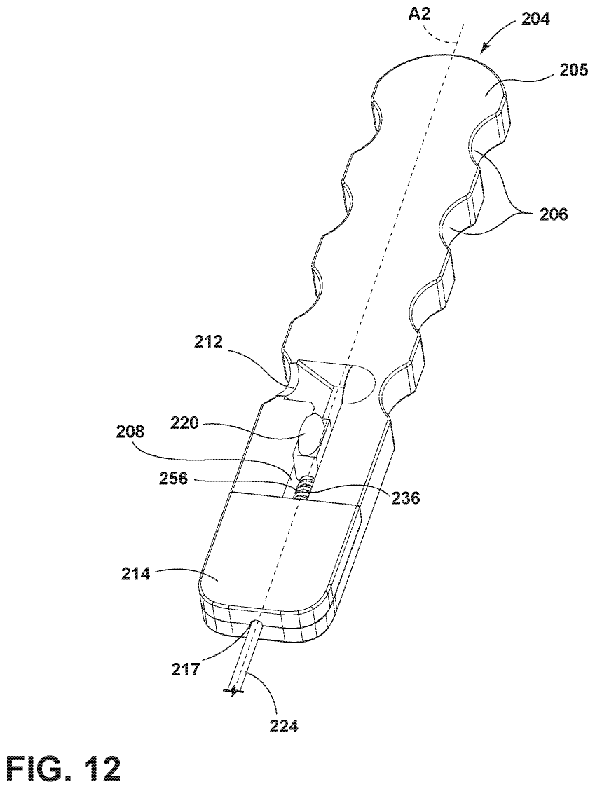

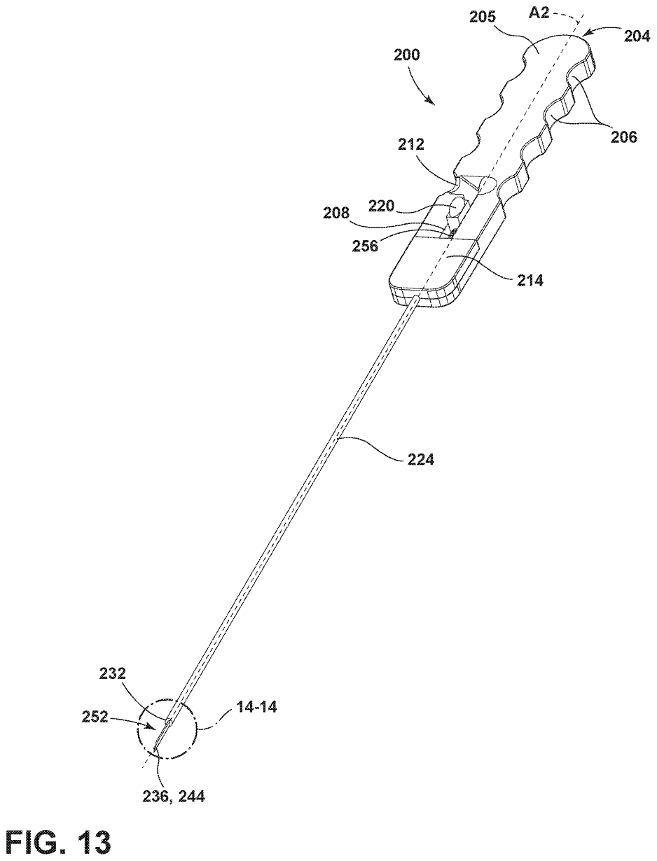

With reference to FIGS. 9-14, the movement of the selector member 220 and the rod 236 will be further discussed. The selector member 220 is movable between a locked state (FIG. 9) and an unlocked state (FIGS. 10 and 12). In the locked state, the selector member 220 is received in the second recess 212. In the unlocked state, the selector member 220 is received in the first recess 208 and is out of the second recess 212. The selector member 220 is considered to be in the locked state in the second recess 212 because the selector member 220 is incapable of moving axially along the longitudinal axis A2 because the main body 205 blocks movement of the selector member 220. When the selector member 220 is in the unlocked state, the selector member 220 is movable along the longitudinal axis A2 between a deployed position (FIG. 12) and an undeployed position (FIGS. 9 and 10). FIG. 11 illustrates the blade 252 in the undeployed position (FIGS. 9 and 10). In the deployed position, the selector member 220 is disposed closer to the channel 217 than when the selector member 220 is in the undeployed position and the blade 252 is not covered by the sheath 224 such that the blade 252 can engage tissue, such as a stricture. Specifically, as the selector member 220 is moved axially along the longitudinal axis A2 toward the channel 217, the rod 236 moves with the selector member 220 since the first end 240 of the rod 236 is coupled to the selector member 220. The blade 252 emerges from the sheath 224 due to the rod 236 moving relative to the handle 204. That is, the sheath 224 stays fixed relative to the handle 204. The spring 256 is configured to bias the selector member 220 to the undeployed position (FIG. 10).

FIG. 14 illustrates the blade 252 disposed outside the sheath 224. In other words, FIG. 14 illustrates the selector member 220 in the deployed position. The blade 252 is integrated with the rod 236 and is a one-sided blade with a true edge 260 and a false edge 264. In the illustrated construction, the blade includes bevels 266 adjacent to the true edge 260. In the illustrated construction, the bevels 266 are straight. In some constructions, the bevels 266 are curved. In the illustrated construction, the blade 252 defines a length L3 measured in a direction parallel to the longitudinal axis A2. In the illustrated construction, the length L3 is 5 millimeters. In some constructions, the length L3 is between 2 and 8 millimeters. The true edge 260 defines a radius that is less than or equal to 50 micrometers. In some constructions, the blade 252 is formed from wire electrical discharge machining. In some constructions, the blade 252 is formed from femtosecond/ultrafast laser ablation. In some constructions, the blade 252 is formed from CNC precision profile grinding. In some constructions, the blade 252 is sharpened and edge refined by electrochemical grinding or precision mechanical honing/lapping. In some constructions, the blade 252 is surface finished by electropolishing and passivation. Electropolishing produces a surface roughness of less than 0.1 micrometers, removes nickel-rich surface layers, and improves fatigue life. Passivation ensures a stable, biocompatible titanium-dioxide surface.

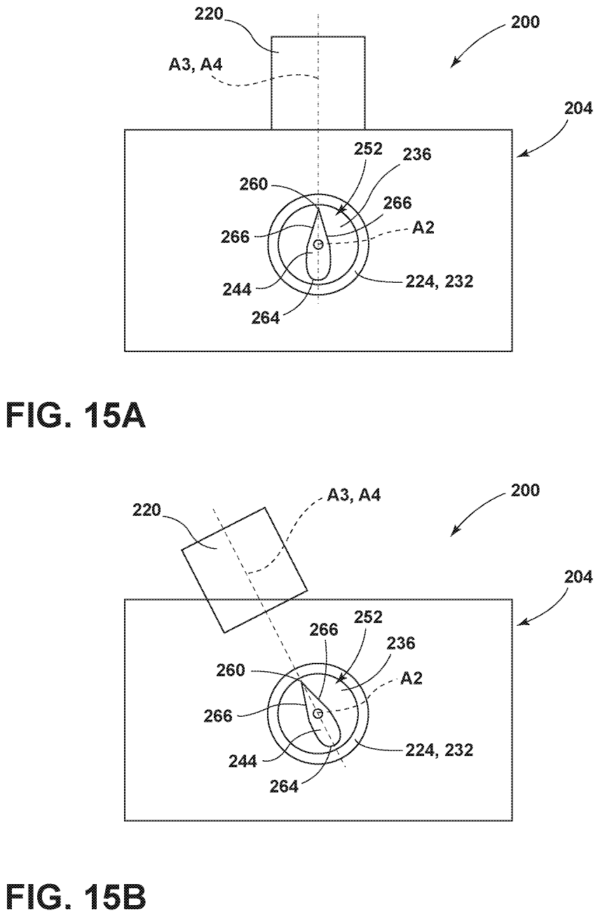

FIGS. 15A and 15B illustrate a schematic view of the surgical blade assembly 200. The true edge 260 defines a blade axis A3 that extends therethrough and is perpendicular to the longitudinal axis A2. The selector member 220 defines a selector member axis A4 that extends through and is perpendicular to the longitudinal axis A2. The true edge 260 is aligned with the selector member 220. In other words, the blade axis A3 and the selector member axis A4 are radially aligned relative to the longitudinal axis A2. That is, the selector member 220 provides an indicator of the position of the true edge 260 since the axes A3, A4 are aligned. FIG. 15A illustrates the selector member in the unlocked state. FIG. 15B illustrates the selector member in the locked state.

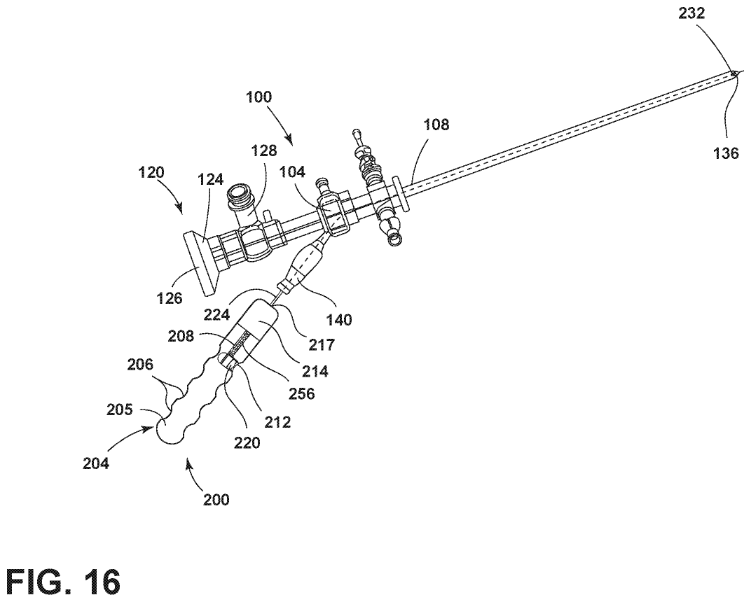

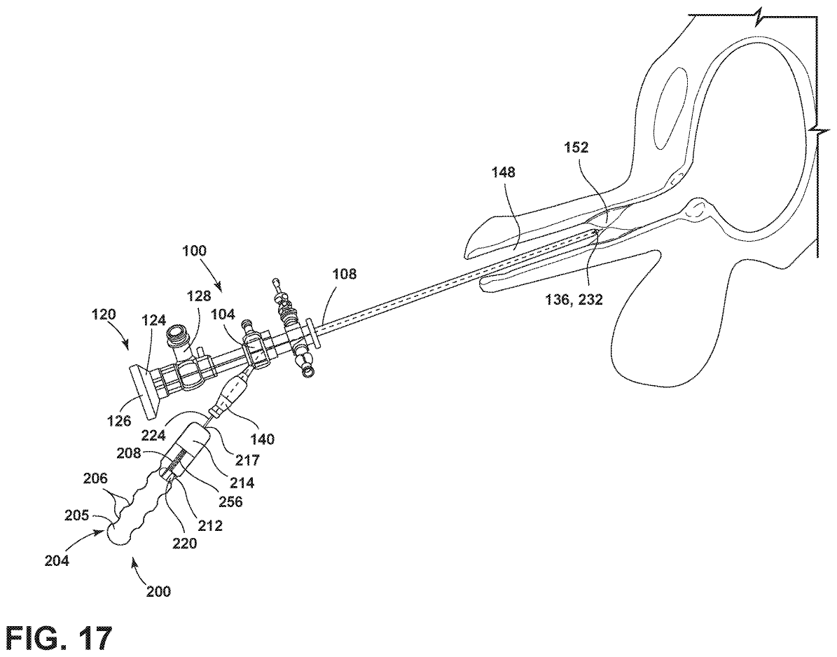

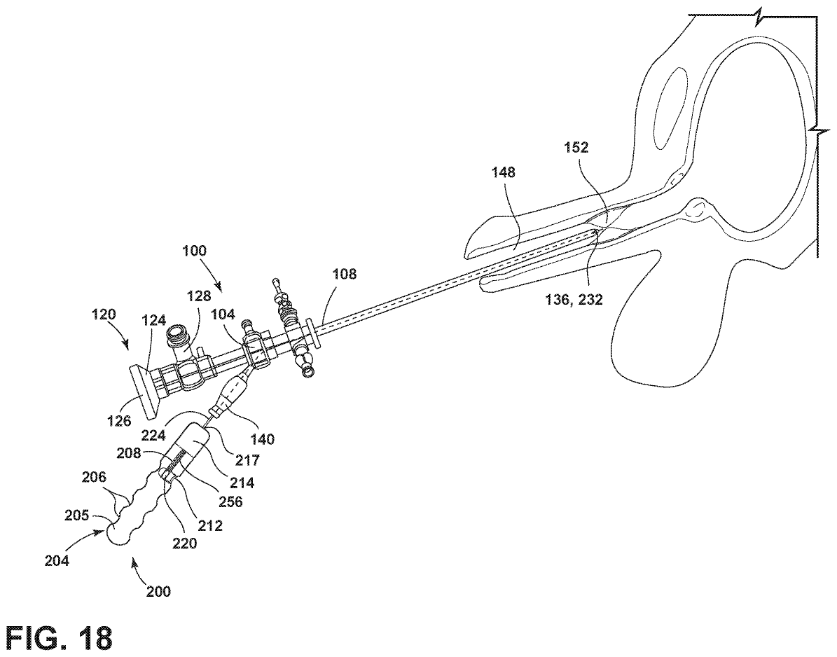

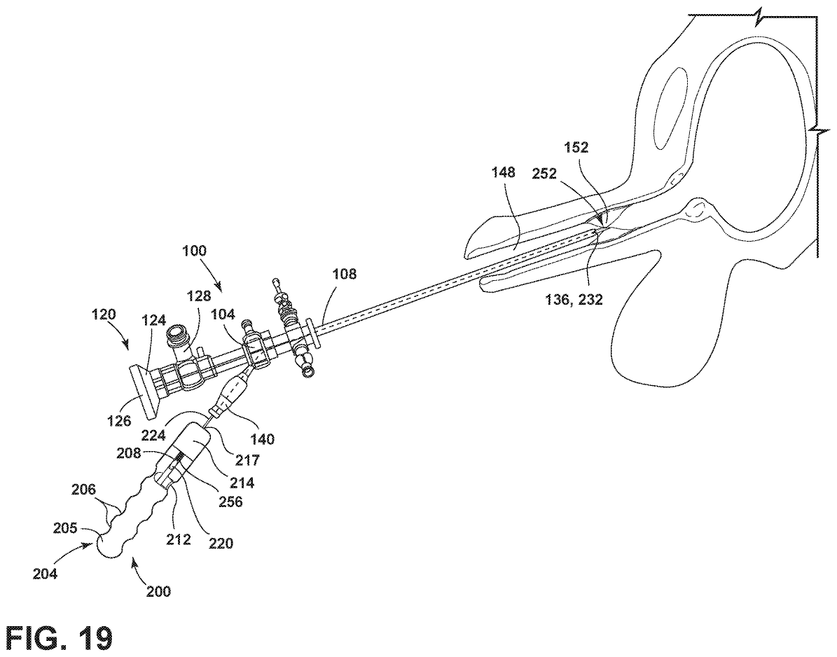

FIGS. 16-19 illustrate using the surgical blade assembly 200 with the cystoscope 100. First, the selector member 220 is set to the locked state such that the blade 252 is not deployed while inserting a portion of the surgical blade assembly 200 into the cystoscope 100. The sheath 224 of the surgical blade assembly 200, illustrated schematically, is inserted into the inlet port 140, which leads into the working channel 116 of the sheath 108. The surgical blade assembly 200 is positioned in the cystoscope 100 such that the second end 232 of the sheath 224 is flush with the beak 136. With reference to FIG. 17, the sheath 108 is inserted into the urethra 148 of the patient. The healthcare provider can identify the stricture 152 in the urethra 148 through the ocular window 126. After the stricture 152 is identified, the sheath 108 can be positioned to face the stricture 152. The selector member 220 can be moved from the locked state (FIG. 17) to the unlocked state (FIG. 18). After determining that the sheath 108 is in the correct position, the healthcare provider can move the selector member 220 from the undeployed position (FIG. 18) to the deployed position (FIG. 19) to deploy the blade 252 from the sheath 224. Since the second end 232 of the sheath 224 is flush with the beak 136, the blade 252 is not protected by the sheath 108. Accordingly, the blade 252 engages the stricture 152. In some instances, the handle 204 can be moved back and forth in the inlet port 140 while the selector member 220 is in the deployed position such that the blade 252 is moved relative to the cystoscope 100 to provide additional axial movement in the urethra 148. In other words, the sheath 224 can be moved relative to the sheath 108.

Although the invention has been described in detail with reference to certain preferred embodiments, variations and modifications exist within the scope and spirit of one or more independent aspects of the invention as described.

Various features of the invention are set forth in the following claims.

What is claimed is:

1. A surgical blade assembly for cutting a urethral stricture, the surgical blade assembly comprising:
a handle defining a longitudinal axis that extends through the handle, the handle including a first recess that extends in a direction parallel to the longitudinal axis and a second recess extending from the first recess;
a selector member received by the handle, the selector member movable between a locked state in which the selector member is received in the second recess and an unlocked state in which the selector member is received in the first recess;
a sheath coupled to the handle;
a rod including a first end and a second end opposite from the first end, the first end coupled to the selector member for movement therewith, the rod at least partially surrounded by the sheath; and
a blade disposed on the second end of the rod, the blade selectively covered by the sheath.

2. The surgical blade assembly of claim 1, further comprising a spring disposed in the first recess and surrounding a portion of the rod, the spring configured to bias the selector member.

3. The surgical blade assembly of claim 2, wherein in the unlocked state, the selector member is movable along the longitudinal axis between a deployed position and an undeployed position, wherein in the deployed position, the selector member is disposed closer to an end of the handle that receives the rod than in the undeployed position and the blade is not covered by the sheath.

4. The surgical blade assembly of claim 3, wherein the spring is configured to bias the selector member to the undeployed position.

5. The surgical blade assembly of claim 1, wherein the rod is constructed from a nickel and titanium alloy.

6. The surgical blade assembly of claim 1, wherein the blade and the rod are formed from a single piece of material.

7. The surgical blade assembly of claim 1, wherein the rod defines a diameter of between 0.6 millimeters and 3.5 millimeters.

8. The surgical blade assembly of claim 1, wherein the blade is a one-sided blade with a true edge and a false edge.

9. The surgical blade assembly of claim 1, wherein the handle includes a main body and a removable portion coupled to the main body, wherein the handle defines a channel that receives the sheath such that the sheath is fixed relative to the handle, wherein the channel is defined between the removable portion and the main body.

10. The surgical blade assembly of claim 9, wherein the rod passes through the channel and is configured to move therein in response to movement of the selector member along the longitudinal axis.

11. The surgical blade assembly of claim 1, wherein the rod defines a length measured from the first end to the second end in a direction parallel to the longitudinal axis, wherein the length is between 235 millimeters and 635 millimeters.

12. The surgical blade assembly of claim 1, wherein the handle includes a plurality of grooves.

13. The surgical blade assembly of claim 1, wherein the sheath is constructed from polyether block amide.

14. The surgical blade assembly of claim 1, wherein the blade includes a true edge that defines a blade axis that extends centrally through the true edge, the blade axis being perpendicular to the longitudinal axis, wherein the selector member defines a selector member axis that extends centrally therethrough, the selector member axis being perpendicular to the longitudinal axis, wherein the blade axis and the selector member axis are radially aligned relative to the longitudinal axis.

15. A method for cutting a urethral stricture, the method comprising:
inserting a sheath of a surgical blade assembly through an inlet port and into a working channel of a cystoscope, the surgical blade assembly including a handle coupled to the sheath and a blade disposed in the sheath and selectively covered by the sheath;
inserting a sheath of the cystoscope into a urethra of a patient;
identifying the stricture in the urethra through an ocular window of the cystoscope; and
deploying the blade from the sheath of the surgical blade assembly such that the blade engages the stricture.

16. The method according to claim 15, wherein the step of inserting the sheath of the surgical blade assembly into the working channel of the cystoscope occurs before the step of inserting the sheath of the cystoscope into the urethra of the patient.

17. The method according to claim 15, wherein the step of deploying the blade from the sheath of the surgical blade assembly such that the blade engages the stricture further includes moving a selector member from a locked state to an unlocked state and moving the selector member along a longitudinal axis of the handle from an undeployed position to a deployed position in which the blade is not surrounded by the sheath.

18. The method according to claim 15, further comprising moving the handle relative to the cystoscope when the blade is deployed from the sheath such that the blade moves axially in the urethra.

19. The method according to claim 15, further comprising aligning an end of the sheath of the surgical blade assembly with an end of the sheath of the cystoscope such that the ends are flush.

20. A surgical blade assembly configured to be partially received within a working channel of a cystoscope, the surgical blade assembly comprising:
a handle defining a longitudinal axis that extends centrally through the handle;
a selector member received by the handle, the selector member movable relative to the handle along the longitudinal axis;
a sheath fixedly coupled to the handle;
a rod at least partially surrounded by the sheath, the rod including a first end and a second end opposite from the first end, the first end coupled to the selector member for movement therewith; and
a blade formed on the second end of the rod, the blade selectively covered by the sheath, wherein the blade and the rod are formed from a single piece of material.
